(19) United States
(12) Patent Application Publication
(10) Pub. No.: US 2013/0252288 A1
(43) Pub. Date: Sep. 26, 2013

(54) ENANTIOSELECTIVE REDUCTION

(71) Applicants: Stefaan Marie André De Wildeman, Maasmechelen (BE); Henricus Martinus Maria Straatman, LG Horst (NL); Gerardus Karel Maria Verzijl, AR Well (NL); Linda M. Vermote, HB Sittard (NL); Andreas Hendrikus Maria De Vries, CR Maastricht (NL)

(72) Inventors: Stefaan Marie André De Wildeman, Maasmechelen (BE); Henricus Martinus Maria Straatman, LG Horst (NL); Gerardus Karel Maria Verzijl, AR Well (NL); Linda M. Vermote, HB Sittard (NL); Andreas Hendrikus Maria De Vries, CR Maastricht (NL)

(21) Appl. No.: 12/668,082

(22) PCT Filed: Jul. 11, 2008
(86) PCT No.: PCT/EP08/59143

§ 371 Date: Jun. 1, 2010

(30) Foreign Application Priority Data
Jul. 11, 2007 (EP) 07013560.3

(51) Int. Cl. C12P 17/10; C12P 7/26; C12P 7/22; C12P 7/24

(52) U.S. Cl. CPC C12P 17/10; C12P 7/24; C12P 7/26; C12P 7/22
USPC 435/121; 435/147; 435/148; 435/156

(57) ABSTRACT

The invention relates to a process for the preparation of a chiral compound, comprising enantioselectively reducing a carbon-carbon double bond of an α,β-unsaturated compound in a mixture comprising both an E isomer and a Z isomer of the α,β-unsaturated compound, wherein both E isomer and Z isomer are converted in the presence of a hydrogenation catalyst.

The invention relates to a process for the enantioselective reduction of an α,β-unsaturated compound and the use of a compound thus prepared, in the preparation of further compounds.

Examples of enantioselective reduction have been described in scientific literature. For example, Fardelone et al., in J. Mol. Catal. B: Enzymatic 29 (2004) 41-45 describe the enantioselective reduction of a-substituted (E)-cinnamaldehyde mediated by baker's yeast, yielding enantiopure α-substituted-3-phenyl-1-propanol.

Ferraboschi et al., in Tetrahedron:Asymmetry 10(1999) 2639 describe the preparation of 2-methyl-1-alkanols by yeast mediated enantioselective biocatalytic reduction of unsaturated compounds containing an methylene group.

Mano et al., in Plant & Cell Physiology, 43(12):1445-1455 (2002), describe the use of an NADPH:Quinone oxidoreductase to convert an alkenal in a aldehyde, using NADPH as a cofactor.

Hall et al., in Angewandte Chemie 2007, 46, 3934-3937, describe the asymmetric reduction of C═C bonds of several unsaturated compounds by an enoate reductase.

In the processes described in the literature referred to above, only the conversion of E-isomers is shown.

The inventors realised that it would be desirable to provide a process wherein both E and Z isomers are converted to a desired enantiomer, whereby a wider substrate spectrum is obtained.

Surprisingly, the inventors found it to be possible to convert both the E and the Z isomer of an α,β-unsaturated compound into a chiral compound by using a specific catalyst, e.g. a hydrogenation catalyst or by using a catalyst, e.g. a hydrogenation catalyst, under specific reaction conditions.

Thus, the invention relates to a process for the preparation of a chiral compound, comprising enantioselectively converting a carbon-carbon double bond of an α,β-unsaturated compound in a mixture comprising both an E isomer and a Z isomer of the α,β-unsaturated compound, wherein both E isomer and Z isomer are converted in the presence of a catalyst, e.g. a hydrogenation catalyst.

The compound obtained after the enantioselective reduction of the α,β-unsaturated compound contains at least one stereogenic centre. In particular the compound may comprise one, two or more stereogenic centres. The compound obtained may be a racemic mixture, a diastereomeric mixture, or be scalemic i.e. enantio-enriched (i.e. one of the enantiomers is formed in excess of the other but not to the exclusion of the other) or enantiopure (i.e. essentially only one of the enantiomers is formed). Thus, the compound may be the 2S, 3S diastereomer, 2S, 3R diastereomer 2R, 3S diastereomer 2R, 3R diastereomer or a mixture thereof.

The term “enantioselective” is used to indicate that one of the enantiomers is formed in excess of the other, or even to the exclusion of the other. (The latter often being referred to as enantiospecific.) Thus, in an enantioselective process of the invention the Z isomer and the E isomer are both preferentially converted into the same enantiomer. Usually, the enantioselectivity is such that one of the enantiomers is formed with enantiomeric excess (ee) of at least 40%, in particular of at least 50% more in particular of at least 60%, even more in particular of at least 70%. Preferably, the enantioselectivity is such that one of the enantiomers is formed with an ee of at least 80%, or of at least 85%. The ee for one of the enantiomers may in particular be about 90% or more, or about 95% or more. Enantiomeric excess of an enantiomer (enantiomer 1) is a measure for how much of one of the enantiomers is present compared to the corresponding enantiomer (enantiomer 2) and is defined as follows.

ee=[Cₑₙₐₙₜᵢₒₘₑᵣ₁−Cₑₙₐₙₜᵢₒₘₑᵣ₂]/[Cₑₙₐₙₜᵢₒₘₑᵣ₁+Cₑₙₐₙₜᵢₒₘₑᵣ₂]×100%

Herein “Cₑₙₐₙₜᵢₒₘₑᵣ₁” is the molar concentration of the enantiomer of which ee is determined and “Cₑₙₐₙₜᵢₒₘₑᵣ₂” is the molar concentration of the corresponding enantiomer.

As the invention allows conversion of both an E isomer and a Z isomer of an unsaturated compound to a desired enantiomer, the conversion of an isomeric mixture of an E isomer and a Z isomer towards a desired enantiomer is higher than in the known processes wherein only one of the isomers would be converted if such known process would be applied to such a mixture of the E isomer and the Z isomer. It has been found possible to choose conditions such that both E isomer and Z isomer are converted with a good yield for a desired enantiomer. At least for some compounds it has been found possible to substantially completely convert both the E isomer and the Z isomer. As used herein, a conversion is in particular considered substantially complete for a specific isomer if at least 90% of that isomer, in particular at least 95% of that isomer is converted to the desired chiral compound.

Thus, if desired, a product may be obtained which may be used, e.g. in a further reaction for preparing a pharmaceutical compound or an intermediate for a pharmaceutical compound, without further purification or after a simpler purification than needed for a product obtained by a known process to obtain a product of similar purity. Moreover, an extra purification step may lead to product loss, and thus reduced overall yield of the product.

The mixture comprising the E isomer and the Z isomer usually has a molar ratio of Z isomer to E isomer of 1:99 to 99:1. In particular, the molar ratio of Z isomer to E isomer may be in the range of 5:95 to 95:5, preferably in the range of 10:90 to 90:10, in particular of 20:80 to 80:20, more in particular of 25:75 to 75:25. A process of the invention offers in particular an advantage in that the Z isomer also can be converted enantioselectively, which makes a method of the invention also particularly interesting in case the mixture which is to be converted comprises a high amount of Z isomer, such as mixture with a molar ratio of Z isomer to E isomer at least 50:50.

In particular an α,β-unsaturated compound represented by Formula 1

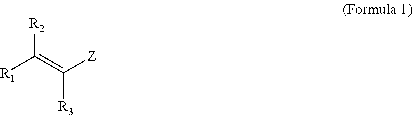

and its corresponding isomer may be converted. Herein Z is an electron withdrawing group, and each of R₁, R₂ and R₃ are independently selected from the group of H, halogen atoms and hydrocarbons, which hydrocarbons optionally comprise one or more heteroatoms. Any combination of two of the moieties R₁, R₂, R₃ and Z are optionally interconnected to form a ring structure, for instance, R₁ and R₂ may form a ring structure together, R₂ and R₃ may form a ring structure together or R₃ and Z may form a ring structure together.

In general, in order to provide an E isomer and a Z isomer R₁ should be different from R₂ and R₃ should be different from Z.

In particular, Z may be selected from the group of —CN, —NO₂, —SO₂, —PO₃ and —(C═O)Q, wherein Q is selected from the group of H, halogen atoms, —OH,;.amine groups; and hydrocarbon groups, in particular comprising between 1 and 12 C-atoms, which hydrocarbons optionally comprise one or more heteroatoms; or —OR wherein R is a hydrocarbon moiety in particular selected from alkyl, alkoxy-alkyl, alkenyl, alkenyl.

Within the context of the present invention the term “hydrocarbon” is meant to include substituted and unsubstituted hydrocarbons, hydrocarbons with one or more heteroatoms (such as S, N, O, P, Cl, Br, F, I, Si) and hydrocarbons without heteroatoms, unless specifically mentioned otherwise. The hydrocarbon may comprise a linear or a branched structure, in particular a linear or branched alkyl, alkenyl or alkynyl, which may optionally be linked to the α,βcarbon-carbon double bond via a heteroatom, for instance O, S, N or Si (to form an alkoxyl, sulphoxyl, amine or silyl respectively). The hydrocarbon may comprise one or more rings, which rings optionally contain one or more heteroatoms, in particular one or more heteroatoms selected from N and O. The ring(s) may be aliphatic (cycloalkyl) or aromatic (aryl). The hydrocarbon, such as the optionally substituted alkyl, optionally substituted cycloalkyl, optionally substituted aryl or optionally substituted alkylaryl, may in particular comprise 1-20 carbon atoms, more in particular 1 to 12 or 1 to 6 carbon atoms. In case of the hydrocarbon comprises a ring, the number of carbon atoms usually is at least 3.

In case the hydrocarbon group comprises one or more heteroatoms, the hydrocarbon group may be attached to the C═C group shown in formula via a carbon or directly via a heteroatom, for instance the hydrocarbon group may be an alkoxy group, an amide group, an organo-silicon group (for instance an trialkylsilyl) or a sulphoxy group.

The hydrocarbon may comprise one or more rings, which optionally contain one or more heteroatoms, in particular one or more heteroatoms selected from N and O. The ring may be aliphatic (cycloalkyl) or aromatic (aryl). The hydrocarbon may in particular comprise 1-20 carbon atoms, more in particular up to 12 or up to 6 carbon atoms. In case of the hydrocarbon comprises a ring, the number of carbon atoms usually is at least 3.

In a preferred process, the electron withdrawing group Z is represented by —(C═O)Q, wherein Q is H, an unsubstituted or substituted alkyl or an unsubstituted or substituted alkoxy. The alkyl or alkoxy may in particular be substituted with —OH or a halogen atom. The alkyl or alkoxy may in particular contain 1-20 carbons, more in particular 1-12 carbons, even more in particular 1-6 carbons.

In an embodiment R₁ is an optionally substituted aryl, preferably an optionally substituted phenyl. In a preferred example of this embodiment, the α,β-unsaturated compound is represented by Formula 2

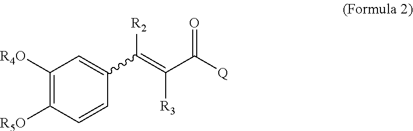

wherein the bond between the aryl group and the α,β unsaturation is in the E or Z position, and wherein R₂ is H; R₃ is an alkyl group comprising between 1 and 12 C-atoms; R₄ and R₅ are independently selected from H, hydrocarbon groups and oxygen protective groups; and Q is selected from the group of H; halogen atoms; —OH; or —OR, wherein R is a hydrocarbon moiety in particular selected from alkyl; and hydrocarbons, which hydrocarbons optionally comprise one or more heteroatoms. Preferably Q is H.

Such a compound may for instance be used as an intermediate compound for a pharmaceutical.

In a specific embodiment R₄ and R₅ are linked to form a ring structure.

Within the context of the present invention the term “oxygen protective group” is used to describe any group suitable to protect an oxygen of an alcohol against an undesired reaction. In particular, the oxygen protective group may be selected from the group of tosylate, mesylate, benzoylate, benzoate, tri-hydrocarbon-silyl, e.g. trimethyl-silyl, and organic acid residues, such as acetate.

In an embodiment, a process of the invention comprises conversion of an isomeric mixture of an aliphatic alkenal, preferably a citral mixture, comprising both the E isomer (geranial) and the Z isomer (neral) of citral.

The catalyst may be a catalyst that is capable of enantioselectively converting both the E isomer and the Z isomer into the desired enantiomer.

The process may further comprise the use of a combination of catalysts, wherein a first catalyst (preferentially) catalyses the conversion of the E isomer and a second catalyst (preferentially) catalyses the conversion of the Z isomer.

However, the inventors came to the surprising insight that it is also possible to accomplish conversion of both isomers by using a catalyst which—at least until now—has been shown substantial conversion of either the Z isomer or the E isomer, thus, not reaching yields higher than the maximum theoretical yield based on the starting amount of either the E- or the Z-isomer. The inventors surprisingly found that in the presence of such a catalyst and a specific reagent, conversion of both isomers can be achieved. Without being bound by theory, it is contemplated that the specific reagent facilitates isomerisation, i.e. transition of Z isomer into the E isomer and/or transition of the E isomer into the Z isomer. It contemplated that such reagent reacts with the isomers in a Michael addition and that thereafter a retro-Michael addition reaction takes place, whereby the transition into the other isomer may occur.

It is in particular surprising that said specific reagent can be used in the presence of the catalyst, without unacceptably detrimentally affecting the activity of the catalyst. Thus, when using said reagent, there is no need to first transform the isomer that is not or to a lesser extent converted by the catalyst into the active (or more active) isomer, and thereafter in an isolated process step, to reduce the isomer. Thus, the process can advantageously take place in a one-pot process, while still allowing a maximum theoretical yield of the desired chiral compound of 100%, based on the amount of starting isomers. Apart from the fact that such process can be employed making use of a simpler reactor system, the isomerisation may take place more efficiently, e.g. quicker and/or more complete.

The process according to the invention is carried out under isomerising conditions. Isomerising conditions are defined as conditions under which the E and Z isomers of the starting material are converted into each other. Isomerising conditions may be obtained in a variety of ways.

Such conditions may be provided by carrying out the reduction in the presence of a compound capable of participating in a Michael addition and retro-Michael addition with the unsaturated compound. Preferably such compound is selected from the group of thiols, including thio-alcohols, alkoxythiols etc; halogens; secondary amines and tertiary amines.

The thiol may for instance be an alkane thiol, e.g., ethane thiol, propane thiol or butane thiol, or an aryl thiol, e.g. thiophenol.

In particular, a thio-alcohol may be used as a compound capable of participating in a Michael addition and/or retro-Michael addition. In a preferred embodiment the thio-alcohol is selected from dithiothreythol, 2-hydroxy-1-ethanethiol, hydroxypropane thiol and hydroxybutane thiol.

Of the halogens used as a compound capable of participating in a Michael addition and/or retro-Michael addition, bromine and iodine, and in particular iodine, are preferred for their handling properties.

The amines used as a compound capable of participating in a Michael addition and/or retro-Michael addition may in particular be selected from cyclic secondary amines, dialkylamines and trialkylamines, including substituted cyclic amines, di- and trialkylamines. The alkyl groups of these amines may in particular be a C1-C6 alkyl, more in particular an ethyl or propyl. Pyrrolidine is an example of a suitable cyclic amine.

If used, the compound capable of participating in a Michael addition and/or retro-Michael addition may be used in a catalytic amount, although higher amounts may be used. The molar ratio of said compound capable of participating in a Michael addition and/or retro-Michael addition to the isomers can be chosen within wide limits. Usually the ratio may be within the range of 0.00001:1 to 100:1, preferably in the range of 0.001:1 to 10:1, more preferably 0.01:1 to 0.5:1.

In an embodiment, isomerising conditions comprise exposure to light promoting isomerisation.

The catalyst used in accordance with the invention may in principle be any catalyst capable of catalysing the reduction of the α,β unsaturation of at least one of the isomers.

In an embodiment, a homogeneous hydrogenation catalyst is used. Homogeneous asymmetric hydrogenation catalysts are known per se. Such catalysts are, for example, described by John M. Brown in E. Jacobsen, A. Pfaltz, H. Yamamoto(Eds.), Comprehensive Asymmetric Catalysis I to III, Springer Verlag,1999, pages 121 to 182. The homogenous hydrogenation catalyst may in particular be selected for its capability of selectively reducing the α,β unsaturation over any other C═C in the α,β unsaturated compound in a mixture comprising both an E isomer and a Z isomer, if said compound comprises one or more other C═C bonds.

Particularly suitable metal (ion) complex are selected from the group of rhodium complexes, ruthenium complexes, iridium complexes and platinum complexes.

Ligands may be selected from inorganic ligands, e.g. , CO, halogenides (chloride, fluoride, bromide, iodide), NH₃ etc, and organic ligands, such as organic acids or anions thereof, e.g. citrate, acetate etc.

Optionally, in particular in a method wherein use is made of a metal (ion) complex, the hydrogenation may be carried out in the presence of a ligand promoting asymmetric hydrogenation. In particular, chiral ligands are suitable to promote asymmetric hydrogenation. The ligand may form a complex with the metal (ion). Suitable ligands are, e.g., described in WO 02/02487.

Suitable hydrogen sources when using a homogeneous hydrogenation catalyst, are known in the art. Suitable sources are hydrogen, and compounds capable of delivering hydrogen, such as alcohols, carboxylic acids, and amines.

Preferably, a biocatalyst is used. The biocatalyst may in particular comprise an enzyme, which enzyme may be employed isolated from an organism or inside an organism expressing the enzyme. In particular a micro-organism expressing the enzyme may be used. The biocatalyst may be dissolved, suspended or dispersed in a reaction medium or immobilised on a carrier. The organism expressing the enzyme may be a naturally occurring organism or a genetically modified (transgenic) organism. The organism may in particular be a micro-organism, more in particular a micro-organism selected from bacteria, fungi and yeasts.

Biocatalysts usually show advantageous selectivity for an α,β unsaturation in a compound, also if one or more other C═C bonds are present in the compound.

Suitable hydrogen sources when using a biocatalyst, are known in the art. Suitable sources in particular include NADPH (Nicotinamide adenine dinucleotide phosphate) and NADH (Nicotinamide adenine dinucleotide).

In particular, an enzyme having oxidoreductase activity (EC class 1) may be used. Preferably a reductase is used, of which ene reductases (E.C.1.3.1.x) are particular preferred. In particular, the ene reductase may be classified as groups containing enal reductases, enone reductases and enoate reductases dependent on the main substrates being converted by each of the respective ene reductase groups. In principle, any such enzyme may be used, e.g., any enzyme described in the above identified publications.

Preferred reductases include old yellow enzymes [EC 1.3.1.x]. Old yellow Enzymes may also be classified under E.C.1.6.99.1. However, in the framework of the invention it is in particular the ene-reductase activity that is of interest, hence, the classification of E.C.1.3.1 is given, but OYE enzymes are expressly included in the enzymes suitable for the process according to the invention. Particular preferred are such enzymes from yeasts:
(OYE Saccharomyces carlsbergensis (GENBANK/X53597)
OYE2 Saccharomyces cerevisiae (GENBANK/L06124)
OYE3 Saccharomyces cerevisiae (GENBANK/L29279)
HYE1 Hansenula polymorpha (GENBANK/AF486188HYE1)
HYE2 Hansenula polymorpha (GENBANK/AF486188HYE2)), from plants:
(P1 Arabidopsis thaliana (GENBANK/Z49768)) or from mammals:
(LTB4DH Rat (Genbank/NM_{—}138863)).

The enzyme, in particular a reductase, is preferably used in combination with a suitable cofactor regeneration system for the enzyme (reductase) which is known to those skilled in the art. Examples are the use of formate dehydrogenase combined with formate, or the use of glucose dehydrogenase combined with glucose. Catalytic amounts of cofactor generally suffice in these cofactor recycling systems.

A catalyst capable of converting both E isomer and Z isomer is a substrate unspecific catalyst, whereas catalysts capable of only converting E isomer or Z isomer are know as substrate specific. In the framework of the invention, a catalyst is in particular considered substrate specific if the conversion rate for one of the isomers (either E or Z) under process conditions is at least 10 times higher, preferably at least 100 times higher than for the other isomer. Else the catalyst is considered substrate unspecific. At least the substrate specific catalyst is usually employed under isomerising conditions, as described herein.

The enzyme, preferably a reductase can be used isolated (solubilized, suspended or on a carrier) from an organism or in an organism, preferably a bacterium, such as E. coli. If desired, the organism can be genetically modified to include the enzyme from another species. Optionally one or more other enzymes, in particular an enzyme for regenerating the reduced cofactor catalyzing the overall hydrogenation are co-expressed. In a particularly preferred method, a bacterium, such as E. coli, is used with co-expressed reductase and a co-factor regeneration enzyme for the reductase.

Optionally one or more other enzymes which, e.g., can catalyse a further reaction of the chiral compound or catalyse isomerisation, can be co-expressed with the enzyme capable of catalysing hydrogenation.

In an advantageous method of the invention, enantioselective conversion, typically reduction, of the carbon-carbon double bond of the α,β-unsaturated compound and a further modification of the compound, in particular a further modification wherein group Z in a compound according to Formula 1 is converted, takes place in a “one-pot” process. In a “one-pot” process reaction conditions are such that both reduction of the isomers and the further modification take place. In particular, in such process, a hydrogenation catalyst for the reduction (plus any needed reagent, in particular a hydrogen source, and—if desired a regeneration system) and a catalyst for such further modification (and any needed reagent, and—if desired—a regeneration system) are present simultaneously in the reaction medium also comprising the E isomer and the Z isomer of the α,β-unsaturated compound.

The chiral compound produced in accordance with the invention may be a pharmaceutically active compound, an intermediate for a pharmaceutically active compound, an agrochemical or an intermediate for an agrochemical or another useful compound.

The chiral compound may be further converted into a different compound. In an embodiment, a further reducible group of the chiral compound is reduced. Further reducible groups in particular may be selected from the group of keto-groups (to be converted in an hydroxyl-group), aldehyde-groups (to be converted in an hydroxyl-group), nitro-groups (to be converted into an amine) and nitril groups (to be converted in an amine).

In particular, in case the chiral compound comprises an aldehyde- or keto-group it may be converted into a corresponding alcohol. Reduction of the aldehyde- or keto-group can be accomplished chemically or enzymatically. Enzymatic reduction can in particular be done using an alcohol dehydrogenase. Suitable reaction conditions may be based on conditions known in the art.

In a preferred embodiment, the further conversion is carried out using as the α,β-unsaturated compound a compound represented by Formula 2

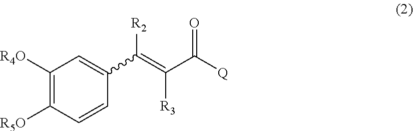

wherein R₂ is H; R₃ is an alkyl group comprising between 1 and 12 C-atoms; R₄ is selected from the group of hydrogen, C₁-C₆ alkyls, C₂-C₆ alkoxyalkyls and oxygen protective groups; R₅ is selected from the group of hydrogen, C₁-C₆ alkyl or an oxygen protective group; Q is H, to provide a compound with formula (3)

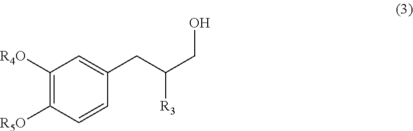

wherein R₃, R₄ and R₅ are as described above for formula (2).

In an embodiment, the conversion of the carbon-carbon double bond according to the invention and the reduction of the electron withdrawing group (such as Z in Formula 1) are carried out in the same reaction medium.

Further conversions are also possible. In an embodiment the compound with formula (3) wherein R₃, R₄ and R₅ are as described above for formula (2), the hydroxyl group is subsequently substituted by a halogen atom, preferably Cl, to form a compound according to Formula (X),

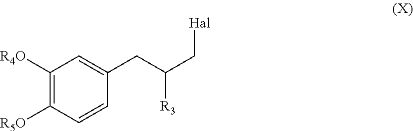

wherein R₃, R₄ and R₅ are as described above for formula (2), and Hal is a halogen atom.

Preferably, for compounds of formula (3) and compound of formula (X) R₃ is 2-propyl, R₄ is 3-methoxypropyl and R₅ is methyl,

In an especially preferred embodiment, enantioselective reduction of the carbon-carbon double bond of the α,β-unsaturated compound and a reduction of a further reducible group is carried out in the presence of a (oxido)reductase, a regeneration system for the (oxido)reductase and an enzyme capable of catalysing the reduction of the further reducible group (which enzyme usually is different from said (oxido)reductase). If desired, a regeneration system for the enzyme capable of catalysing the reduction of the further reducible group may be present, which may be the same or different as the regeneration system for the (oxido)reductase.

Accordingly, the invention also relates to an enzyme-mixture—which may be free of the α,β-unsaturated compound that is to be converted—comprising a (a) (oxido)reductase for reducing a carbon-carbon double bond of an α,β-unsaturated compound, (b) a regeneration system for the (oxido)reductase, (c) an enzyme capable of catalysing the reduction of the further reducible group (other than the carbon-carbon double bond of an α,β-unsaturated compound), and (d) optionally a regeneration system for the enzyme capable of catalysing the reduction of the further reducible group may be present, which may be the same or different as the regeneration system for the (oxido)reductase.

A preferred enzyme mixture of the invention comprises an (a) ene reductase, (b) a cofactor regenerating oxidoreductase and an (c) alcohol dehydrogenase.

The invention further relates to a micro-organism, wherein at least two, preferably at least three of an (a) (oxido)reductase for reducing a carbon-carbon double bond of an α,β-unsaturated compound, (b) a regeneration system for the (oxido)reductase, (c) an enzyme capable of catalysing the reduction of the further reducible group (other than the carbon-carbon double bond of an α,β-unsaturated compound) are expressed, preferably a bacterium or yeast, more preferably E. coli.

Preferably (a) is ene reductase, (b) is a cofactor regenerating oxidoreductase (often a dehydrogenase) and (c) is an alcohol dehydrogenase.

Particularly preferred enzymes for the mixture or expressed in the organism are as identified above and/or in the claims.

In a further embodiment, Z in formula 1 is an NO₂ group. The NO₂ group may be converted into a primary, secondary or tertiary amine. Suitable reaction conditions may be based on conditions known in the art.

In particular a compound according to Formula 3 prepared according to a process of the invention may be used for the preparation of a compound represented by Formula (XVII)

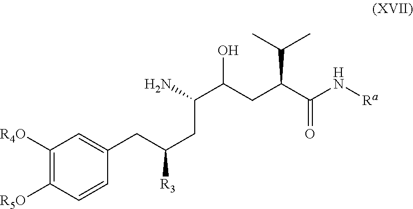

wherein R₃, R₄ and R₅ are as described above for formula (2), R^{a} is H, or a hydrocarbons, which hydrocarbons optionally comprise one or more heteroatoms.

The invention will now be illustrated with the following examples.

### EXAMPLES

### Example 1

a) Preparation of pyrrolidino-3-methylbut-1-ene (enamine)

194 g (2.25 mol) of isovaleraldehyde were diluted in 1115 ml of toluene and cooled, with stirring, to 0° C. To this solution, 190.3 g (2.68 mol) of pyrrolidine, diluted in 185.8 ml of toluene, were added dropwise, so that the reaction temperature did not rise above 5° C. Upon completion of the addition, the reaction solution was stirred for one more hour at 5° C. Thereupon the reaction mixture was heated to room temperature and the water formed was separated completely by extraction with toluene. After this, the solvent was removed through evaporation and the crude product (329.1 g; 95% of the theoretical yield) stored in a refrigerator at 4° C.

b) Reaction of enamine as prepared in Example 1a with 4-methoxy-3-(3-methoxy-propoxy)-benzaldehyde (A1)

222.3 g (0.99 mol) of A1 were diluted with 240 g of 2-propanol. To this solution 321.2 g (2.31 mol) of the enamine were added, with stirring, at room temperature. The reaction mixture was then heated to 80° C. and stirred at this temperature for 50 hours. To remove unreacted A1, the reaction mixture was extracted with 1170 ml of NaHSO₃ (40%) and 1365 ml of water for 30 minutes.

The unreacted enamine was evaporated using a rotary evaporator and entrained with 2-propanol (40 mbar, 50° C.). After an aqueous extraction, 148.4 g of the product aldehyde (51.2%) were obtained.

### Example 2

60 g (394 mmol) of isovanillin were dissolved in 200 ml of DMF and cooled to 0° C. 120 g of Et₃N were added and 63 g (550 mmol) of methane sulfonyl chloride were slowly added dropwise. Thereupon the reaction mixture was extracted with EtOAc and HCl and then rotated to dryness (60° C., 10 mbar). Yield 83 g mesylated isovanillin (92% of the theoretical yield).

83 g (360 mmol) of mesylated isovanillin were dissolved in 250 ml of DMF and 250 ml of toluene and, with stirring, reacted at 60° C. with 90 g (646 mmol) of enamine prepared according to Example 1a.

Thereupon the solvent was extracted using a rotary evaporator (Rotavapor).

Yield 70 g (65% of the theoretical yield).

### Example 3

Method of preparation of 2-(3-(methoxypropoxy)-4-methoxybenzyl)-3-methylbutanal by 2-electron bioreduction of 2-(3-(methoxypropoxy)-4-methoxybenzylidene)-3-methylbutanal with E. coli cells expressing Enone Reductase (ER), adding Glucose Dehydrogenase (GDH from Bacillus megaterium purchased at Jülich Chiral Solutions) for cofactor recycle, yielding highly enantiomerically enriched saturated aldehyde

The example focuses on the production of enantio-enriched saturated aldehyde under isomerising conditions starting from the E/Z mixture of 2-(3-(methoxypropoxy)-4-methoxybenzylidene)-3-methylbutanal. 1,4 dithio-DL-threitol (DTT) is used as isomerisation catalyst.

Conditions:

Atmospheric pressure, 25° C., pH=7.5 (pH adjustment with NaOH)

Ingredients needed:

2-(3-(methoxypropoxy)-4-methoxybenzylidene)-3-methylbutanal (149.4 mg oil, purity=95%, E/Z ratio=74/26), Potassium phosphate buffer 100 mM pH=7.5 (27 ml), NADP⁺ (25 mg),

Cell free extract (prepared via sonification) of E. coli TOP10 cells expressing Enone Reductase P1 from A. thaliana (3 ml cell free extract, equivalent with 230 mg cell wet weight, 25% over-expression of total protein), glucose dehydrogenase (400 units), glucose (200 mg), 1,4 dithio-DL-threitol (DTT, 1 ml of 1M solution in water). All over-expression experiments were carried out following Invitrogen protocols at www.invitrogen.com for Gateway cloning.

Results:

After 24 hr 2-(3-(methoxypropoxy)-4-methoxybenzylidene)-3-methylbutanal conversion was >99%, closing the carbon balance as follows: >90% had been converted to the (R)-enantiomer of the corresponding saturated aldehyde (e.e.=82%), <10% was converted to the corresponding saturated alcohol (due to background ADH activity of E.coli cells).

### Example 4

Method for the preparation of 2-(3-(methoxypropoxy)-4-methoxybenzyl)-3-methylbutanol by 4-electron bioreduction of 2-(3-(methoxypropoxy)-4-methoxybenzylidene)-3-methylbutanal with E.coli cells expressing Enone Reductase (ER), E.coli TOP10 cells expressing Alcohol Dehydrogenase (ADH), adding Glucose Dehydrogenase (GDH from Bacillus megaterium purchased at Jülich Chiral Solutions) for cofactor recycle, yielding highly enantiomerically enriched saturated alcohol.

The example focuses on the production of enantio-enriched saturated alcohol under isomerising conditions starting from the E/Z mixture of 2-(3-(methoxypropoxy)-4-methoxybenzylidene)-3-methylbutanal. 1,4 dithio-DL-threitol (DTT) is used as isomerisation catalyst.

Conditions:

Atmospheric pressure, 25° C., pH=7.5 (pH adjustment with NaOH)

Ingredients needed:

2-(3-(methoxypropoxy)-4-methoxybenzylidene)-3-methylbutanal (151.1 mg oil, purity=95%, E/Z ratio=74/26), Potassium phosphate buffer 100 mM pH=7.5 (27 ml), NADP⁺ (25 mg),

Cell free extract (prepared via sonification) of E.coli TOP10 cells (purchased at Invitrogen) expressing Enone Reductase P1 (3 ml cell free extract, equivalent with 230 mg cell wet weight, 25% over-expression of total protein), cell free extract (prepared via sonification) of E.coli TOP10 cells expressing ADH E7 (1 ml cell free extract, equivalent with 80 mg cell wet weight, 30% over-expression of total protein), glucose dehydrogenase (400 units), glucose (200 mg), 1,4 dithio-DL-threitol (DTT, 1 ml of 1M solution in water). All over-expression experiments were carried out following Invitrogen protocols at www.invitrogen.com for Gateway cloning.

Results:

After 24 hr 2-(3-(methoxypropoxy)-4-methoxybenzylidene)-3-methylbutanal conversion was >99%, almost closing the carbon balance with the saturated alcohol (4-electron reduced product). As a result, >90% of the almost completely converted substrate had been converted to the (R)-enantiomer of the corresponding saturated alcohol (e.e.=82%).

### Example 5

The effect of an isomerising catalyst in the 2-electron bioreduction of 2-(3-(methoxypropoxy)-4-methoxybenzylidene)-3-methylbutanal with E.coli cells expressing Enone Reductase (ER) and added glucose Dehydrogenase (GDH from Bacillus megaterium purchased at Jülich Chiral Solutions) for cofactor recycle, yielding enantiomerically enriched corresponding saturated aldehyde.

The example focuses on the effect of an isomerising catalyst in the production of enantio-enriched saturated aldehyde starting from the E/Z mixture of 2-(3-(methoxypropoxy)-4-methoxybenzylidene)-3-methylbutanal.

Conditions:

Atmospheric pressure, 25° C., pH=7.5 (pH adjustment with NaOH)

Ingredients needed:

A) 2-(3-(methoxypropoxy)-4-methoxybenzylidene)-3-methylbutanal (150 mg oil, purity=95%, E/Z ratio=74/26), potassium phosphate buffer 100 mM pH=7.5 (27 ml), NADP⁺ (25 mg),

Cell free extract (prepared via sonification) of E.coli TOP10 cells expressing Enone Reductase P1 (1.2 ml cell free extract, equivalent with 300 mg cell wet weight, 9% over-expression of total protein), glucose dehydrogenase (400 units), glucose (200 mg), 1,4 dithio-DL-threitol (DTT, 1 ml of 1M solution in water, end concentration=30 mM). All over-expression experiments were carried out following Invitrogen protocols at www.invitrogen.com for Gateway cloning.

B) As A, but instead of DTT, Mercapto-ethanol is added (70 mg, end concentration=30mM).

C) As A, but no additive.

Results:

Z-2-(3-(methoxypropoxy)-4-methoxybenzylidene)-3-methylbutanal is not converted if no isomerisation catalyst is added. The Enone Reductase does not accept the Z-isomer.

If DTT or Mercapto-ethanol is added the Z-isomer is converted via isomerisation towards to E-isomer and further by the Enone Reductase to the saturated aldehyde.

1. Process for the preparation of a chiral compound, comprising enantioselectively reducing a carbon-carbon double bond of an α,β-unsaturated compound in a mixture comprising both an E isomer and a Z isomer of the α,β-unsaturated compound, wherein both E isomer and Z isomer are converted in the presence of a catalyst, and wherein the reduction is carried out under isomerising conditions.

2. Process according claim 1, wherein the molar ratio Z isomer to E isomer in the mixture at the start of the process is in the range of 5:95 to 95:5, in particular 10:90 to 90:10, more in particular 20:80 to 80:20.

3. Process according to claim 1, wherein the chiral compound is formed with an enantiomeric excess of at least 50%, in particular of at least 80%, more in particular of at least 90%.

4. Process according to claim 1, wherein the catalyst is a biocatalyst, in particular an enzyme which enzyme may be present in an organism or isolated from an organism, and which enzyme preferably is an oxidoreductase, more preferably an ene reductase.

5. Process according to claim 4, wherein the biocatalyst is an enzyme and reduction is carried out in the presence of a cofactor regeneration system for the enzyme.

6. Process according to claim 4, wherein the enzyme is a substrate unspecific enzyme.

7. Process according to claim 4, wherein the enzyme is a substrate specific enzyme.

8. Process according to claim 4, wherein the enzyme is selected from the group of ene reductases HYE1, HYE2, P1 and LTB4DH.

9. Process according to claim 1, wherein the conversion is carried out in water or an aqueous liquid, optionally comprising a co-solvent.

10. Process according to claim 1, wherein one of the isomers is represented by Formula 1 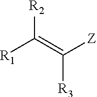
wherein Z is an electron withdrawing group;
and each of R₁, R₂ and R₃ are independently selected from the group of H, halogen atoms and hydrocarbons, which hydrocarbons optionally comprise one or more heteroatoms, and wherein R₁ and R₂ are optionally interconnected to form a ring structure.

11. Process according to claim 10, wherein Z is selected from the group of —CN;
—NO₂; and —(C═O)Q, wherein Q is selected from the group of H; halogen atoms; —OH; —OR, wherein R is a hydrocarbon moiety in particular selected from alkyl, alkoxy-alkyl, alkenyl, alkenyl; amines and hydrocarbons, which hydrocarbons optionally comprise one or more heteroatoms.

12. Process according to claim 11, wherein Z is represented by —(C═O)Q and Q is H or an unsubstituted or substituted alkyl.

13. Process according to claim 10, wherein R₁ is an optionally substituted aryl, preferably an optionally substituted phenyl.

14. Process according to claim 10, wherein the mixture comprises an E and a Z isomer of an aliphatic alkenal, preferably of a mixture of geranial and neral.

15. Process according to claim 1, wherein the α,β-unsaturated compound comprises an electron withdrawing group (such as Z in Formula 1), which is reduced, in particular after said conversion of the carbon-carbon double bond.

16. Process according to claim 15, wherein the electron withdrawing group is selected from the group of keto groups (thereby forming a hydroxyl group), aldehyde groups (thereby forming a hydroxyl group), nitro groups (thereby forming an amine group), and nitril groups (thereby forming an amine group).

17. Process according to claim 16, wherein the α,β-unsaturated compound is a compound represented by Formula 2 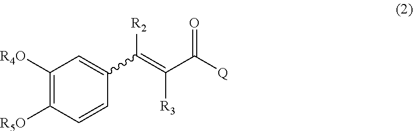
wherein R₂ is H; R₃ is an alkyl group comprising between 1 and 12 C-atoms; R₄ is selected from the group of hydrogen, C₁-C₆ alkyls, C₂-C₆ alkoxyalkyls and oxygen protective groups; R₅ is selected from the group of hydrogen, C₁-C₆ alkyl or an oxygen protective group; Q is H,
to provide a compound with formula (3) 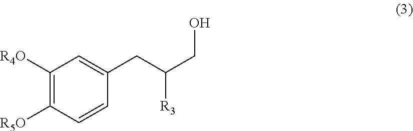
wherein R₃, R₄ and R₅ are as described above for formula (2).

18. Process according to claim 15, wherein the reduction of the carbon-carbon double bond and the reduction of the electron withdrawing group (such as Z in Formula 1) are carried out in the same reaction medium.

19. Process according to claim 17, wherein in a compound according to formula (3) wherein R₃ is 2-propyl, R₄ is 3-methoxypropyl and R₅ is methyl, the hydroxyl group is subsequently substituted by a halogen atom, preferably Cl, to form a compound according to Formula (X), 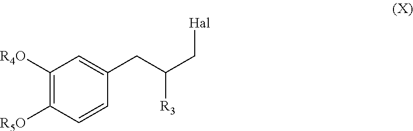
wherein R₃ is 2-propyl, R₄ is 3-methoxypropyl, R₅ is methyl, and Hal is a halogen atom.
